(19) United States
(12) Patent Application Publication
(10) Pub. No.: US 2023/0143699 A1
(43) Pub. Date: May 11, 2023

(54) FOOT NEUROPATHY SPRINGS (FNS)

(71) Applicant: Larry Shane Nolen, Stafford, TX (US)

(72) Inventor: Larry Shane Nolen, Stafford, TX (US)

(21) Appl. No.: 17/412,720

(22) Filed: Aug. 26, 2021

(51) Int. Cl. A61F 5/01; A61F 5/30

(52) U.S. Cl. CPC A61F 5/0113; A61F 5/30; A61F 2005/0197

(57) ABSTRACT

These FOOT NEUROPATHY SPRINGS are devices to help people suffering from genetic diseases or injury that affects a person’s ability to move their feet and that might have a condition known as FOOT DROP. This device helps to correct FOOT DROP, minimize a limp, improve balance, and help propel the person forward by replacing weak muscles with energized springs.

### SUMMARY OF INVENTION

The Foot Neuropathy Springs (FNS) addresses the problems of limited ankle movement associated with Injuries, Diabetes, Charcot-Marie-Tooth Syndrome (a sub-form of muscular dystrophy) and other diseases causing nerve damage to the lower extremities. These dying nerves limits the foot’s ability to dorsiflex or plantarflex the foot — raise or lower. A person limps as there is not enough muscle strength to keep the foot from collapsing at the ankle as they walk. With muscles dying, they also do not have strength to push forward when they take a step. A person cannot raise the foot high enough to keep from dragging their toes and must walk with a gait that forces their knee to be raised higher than normal when walking. There is not enough muscle strength to regulate the foot as it descends to the ground and therefore plops and hits the ground forcefully — this condition is known as Drop Foot. Typical ankle braces immobilizes and limits ankle movements in order to overcome the weakness of the injuries, mostly from an inversion and eversion perspective. When walking, the Foot Neuropathy Springs tighten to keep the ankle from collapsing as one moves forward, the springs load up energy and when the foot moves into a tiptoe/pushing position, releases the energy, and helps propel the person forward. As the bottom spring is flexible, as springs are supposed to be, and does not cover the whole of the foot and stops short of the balls of the feet, it allows the foot to plantar flex as much as possible. As the top spring being flexible, it allows the foot to dorsiflex as needed when navigating inclines as well as provide strength to keep the foot from collapsing. These factors are very helpful in driving a vehicle so as to manipulate the foot pedals. As the spring does cover most of the bottom of the foot, it helps keep the foot level to minimize the drooping of the foot that causes the Drop Foot condition.

### PRIOR ART

Other Ankle Foot Orthotics such as Patent 10,744,019 have bulky design, provide no strength support to prevent limps nor does it provide a way to help propel a person forward. Patent 8,048,012 is also bulky with no strength support to prevent limping or to help propel the person forward. It only helps with the Foot Drop situation. Patent 6,887,217 is non-adjustable, struts connecting the foot plate with the leg support branches would make it very hard to plantar flex or push down if not impossible. This would be very hard to walk downhill. It is also made from very expensive Kevlar. Patent 10,849,778 provide no strength support to prevent limping, no propulsion, and no strength support to navigate inclines. Most braces support the foot from drooping helping prevent Foot Drop and many provide a ridged support of the ankle to provide a person better balance. This current invention is to provide Foot Drop prevention, minimize a limp, provide propulsion forward, be inconspicuous, and be inexpensive as compared to many other AFOs.

### INVENTION BACKGROUND

This invention falls in the A61F 5/0113 category of AFOs — ankle foot orthotics. The FOOT NEUROPATHY SPRINGS (FNS) addresses (1) To stop the collapse of the ankle when walking forward that creates a limp. (2) The need to keep the foot from drooping while walking that leads to stumbling with the loss of muscle activity. (3) The need to keep the ankle joint semi-flexible for plantar or dorsi-flexion so as to navigate stairs, vehicle pedals, and walking hills or various inclines. (4) To help replace the muscle activity that no longer exist to propel the person forward. Current AFOs address some of these conditions, but not all in the same apparatus. (5) The FNS are independent of each other. Various strengths of the springs and lengths can be used to match the requirements of the patient and can be incorporated with existing ankle braces if desired. (6) The FNS can be totally concealed and worn comfortably without having to change shoes sizes or styles and can even be worn with sandals.

### DESCRIPTION OF DRAWINGS

FIG. 1: Depicts movements (A) Dorsiflex and (B) Plantarflex — up and down. These become difficult to do upon command with an axonal peripheral neuropathy such as caused by Diabetes, Charcot-Marie-Tooth Syndrome (a sub-form of muscular dystrophy) or other nerve damaging conditions. Inversion or Eversion movements (C) also become harder to perform as the nerves continue to fail and muscles cannot perform.

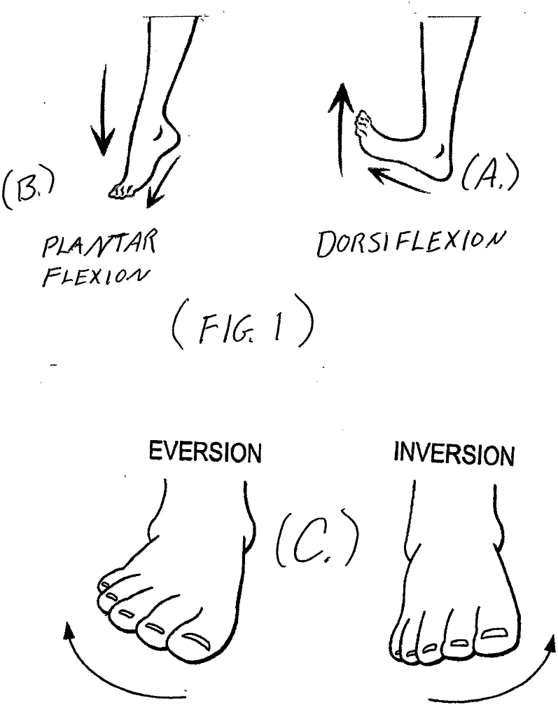

FIG. 2: The pocketed padded sleeves (B) are pulled over the ARCH and over the SHIN above the ankle a few inches. The pockets (C), keeps the FNS (A) in the proper place. (X) Shows the FNS (A), coming up from the bottom of the foot, hugging the heel and continuing up the leg above the ankle a few inches and into the pocket (C) of the elastic padded sleeve (B). (Y) Shows the FNS (A), moving from the end of the arch through the pocket (C) of the elastic sleeve, up the foot (B), then angling up the ankle continuing up the shin a few inches into a pocket (C) of another elastic padded sleeve (B). From a profile view (Z), depicts both FNS (A) as they fit on the foot held in place by the elastic padded pocketed sleeves (B). The top spring (1) moves from the end of the arch, along the foot, up the ankle and against the shin. The bottom FNS (2) runs along the foot short of the balls of the foot, up and around the heel and angles slightly away from the leg ending at near the same height as the bottom spring. The elastic sleeve (B) holds the FNS (A) close the leg under tension.

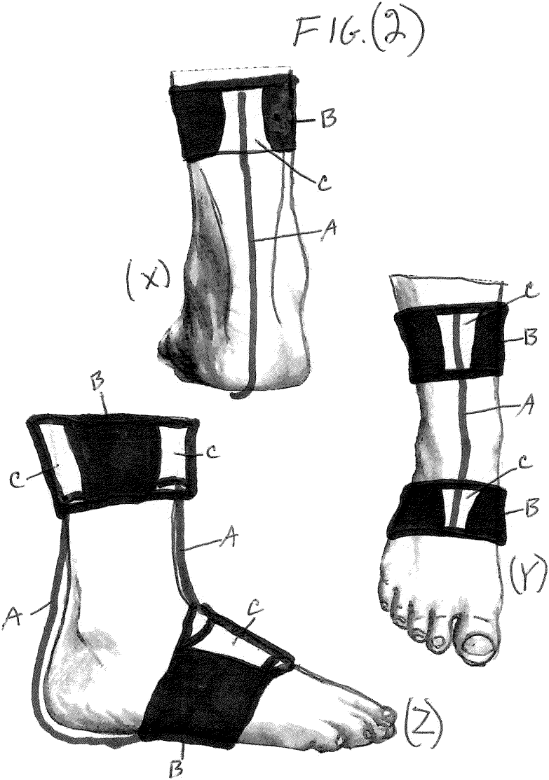

FIG. 3: (Y) Shows both springs closing, building energy (A & B), as the leg moves over the foot while walking forward. (X) Shows both springs opening up releasing the energy as the heel comes up off the floor while walking forward.

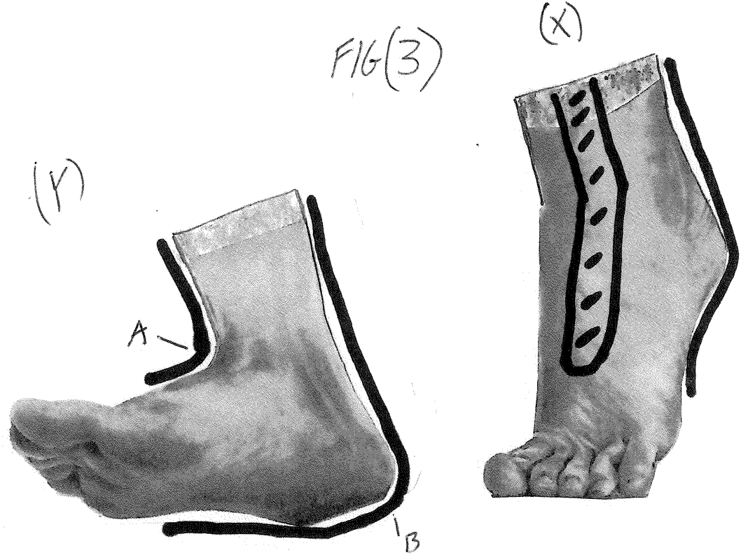

FIG. 4: Shows both FNS (B), fitted in slots at the end of the padded sleeves (A). The padded sleeves (A) are incorporated in a conventional ankle brace permanently, such as stitched in, or semi-permanently, such as with Velcro. (C) depicts a Velcro strap (or could be laces) attached to the rear sleeve (A) so that it could wrap around the leg making sure the springs (B) are held in place against the leg.

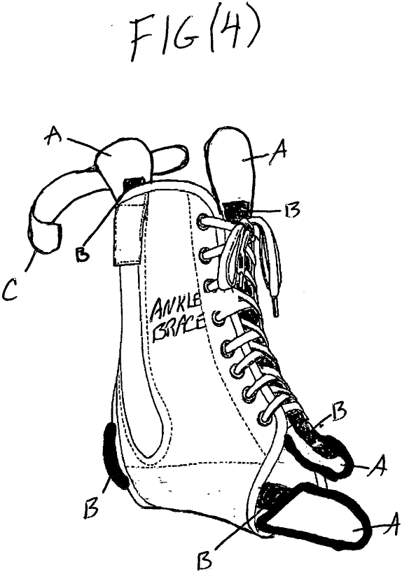

### DETAILED DESCRIPTION

Thin narrow springs, approximately an inch wide, are made to fit in the center of both the top and bottom of either foot. The top spring runs from the end of the arch nearest the toes to the ankle and then angles up the shin several inches. The bottom spring runs from the center of the foot short of the balls of the foot to around the heel and then up the leg angling slightly away from the leg and matches the height of the top spring. Depending on the material of the spring; from glass fiber, carbon fiber, plastic, or mild steel; the thickness can run from sub-millimeter to a couple of millimeters and can also depend on how strong the spring needs to be. Variability is necessary as there is variability in the size of people and nerve damage. The edges are beveled, and the ends rounded for a gentle feel to the body and for being gentle and non-abrasive to the sleeve material that it will fit into. The elastic sleeve holding the springs are made of any fabric of integrity that have slots at both ends of the sleeve in which about 2 inches of the spring will slide into. The sleeve is padded in the areas of where there is sensitivity: (1) the arch (2) the shin bone (3) the heel. The elastic sleeves with the FNS make up a stand-alone brace for the foot, or the springs can be independent and fitted into most existing foot/ankle brace and held in place via Velcro, laces, stitching, or by any other means possible. There is a lace or Velcro strap at the top end of the springs that wraps around the leg and shin securing that part of the springs tightly in place next to the leg while in a slotted pouch of the padded sleeve.

1. Springs conforming to the upper and bottom part of the foot restricts the bend in the ankle when the knee crosses over the ankle when stepping forward, minimizing the ankle collapse.

2. The Springs of claim 1 will release energy as the heel comes up off the ground when stepping forward and help propel the person forward.

3. The springs of claim 1 can be used on either foot or both.

4. The Springs of claim 1 will keep the foot from drooping and minimizes Drop Foot.

5. The Springs of claim 1 will allow the foot to plantar and dorsi flex on a limited basis.

6. The springs of claim 1 fit in elastic pocketed sleeves, padded along the areas of the arch, shin, and heel so that walking can be done in a more natural and comfortable way.

7. The springs of claim 1 can be made to fit permanently or semi-permanently into an existing ankle brace.

8. The springs of claims 6 & 7 can be removed and replaced with various strengths of springs as the conditions of the leg and feet change.
